(19) United States
(12) Patent Application Publication
(10) Pub. No.: US 2025/0050299 A1
(43) Pub. Date: Feb. 13, 2025

(54) METHOD FOR PREDICTING PERFORMANCE DROP OF A COMMERCIAL ALKANE DEHYDROGENATION UNIT AND OPTIMIZING RUN DURATION

(71) Applicant: SABIC GLOBAL TECHNOLOGIES B.V., Bergen op Zoom (NL)

(72) Inventors: Massimiliano Comotti, Jubail (SA); Faysal Benaskar, Jubail (SA); Sami Muteib Al-Mutairi, Dammam (SA); Adel Saud Al-Shafai, Dammam (SA)

(21) Appl. No.: 18/720,844

(22) PCT Filed: Dec. 19, 2022
(86) PCT No.: PCT/IB2022/062482

§ 371 Date: Jun. 17, 2024

(30) Foreign Application Priority Data
Dec. 20, 2021 (EP) 21215894.3

(51) Int. Cl. B01J 19/00; B01J 8/00; C07C 5/00

(52) U.S. Cl. CPC B01J 19/0033; B01J 8/001; C07C 5/00; B01J 2208/00725; B01J 2219/00216; B01J 2219/00218; B01J 2219/00234

(57) ABSTRACT

A process for operating a chemical process includes deriving coefficients for a process performance model from historical feed data and historical production data; formulating the process performance model using the coefficients; determining a predicted change in production of a product of the chemical process using the process performance model; and changing a processing parameter of the chemical process based on economic data and the predicted change in production of the product of the chemical process.

### CROSS REFERENCE TO RELATED APPLICATION

This application claims priority to European Application 21215894.3, filed on Dec. 20, 2021, the content of which is incorporated by reference in its entirety.

### BACKGROUND

Catalyst activity and stability may be assessed by studying catalyst properties and variation of such properties over time under controlled conditions. Catalyst kinetic parameters may be derived from dedicated experiments carried out in a laboratory on small quantities of materials. Kinetics parameters may be used in rate equations to assess product formation and products distribution under well-defined conditions (e.g., temperature, pressure, space velocity). Such experiments and methodology may be applied both to fresh and aged catalysts to link catalyst deactivation coefficient(s) to relevant reaction parameters (e.g., reaction temperature). However, (a) experiments carried out in the laboratory may not address all parameters playing a role in a commercial reactor, (b) some features implemented in the commercial reactor may not be readily reproduced in small scale laboratory experiments, (c) a catalyst owner may not allow a third party to carry out such experiments on its materials, (d) kinetics data may be available on the open literature for similar material(s) but not for commercial catalysts, and (e) temperature may be used to establish rate of catalyst deactivation whereas other parameters may not be readily reproduced on small scale and may play a significant role.

Pilot scale facilities may not accommodate more than a few kilograms of catalyst, and some uncertainties may remain, which may contribute to a potentially inaccurate prediction of performance drop in a commercial unit. This holds true especially for a “Houdry” type dehydrogenation unit of light alkanes, owing to the complexity and the dynamic features of the commercial process.

### BRIEF DESCRIPTION

Prediction of the performance drop and catalyst deactivation in a commercial production unit may enable proper tuning of operation philosophy based both on technical and financial aspects affecting a commercial unit in the timeframe production is carried out. However, all information that may contribute to assessment of performance and performance drop in commercial production units may not be owned, e.g., known, by a single party and it may not be possible to use such information in a synergic manner. The disclosed process provides a tool capable of predicting catalyst performance deactivation and performance drop using process parameters and not information gathered from catalyst kinetics and catalyst properties. Performance drop may not be directly linked to catalyst activity coefficients, as other factors may play a role, for example, the presence of hot spots in reactors affecting the possibility to increase or maintain reactor(s) heat input.

Disclosed herein is a process for operating a chemical process, including deriving coefficients for a process performance model from historical feed data and historical production data; formulating the process performance model using the coefficients; determining a predicted change in production of a product of the chemical process using the process performance model; and changing a processing parameter of the chemical process based on economic data and the predicted change in production of the product of the chemical process.

The above described and other features are exemplified by the following figures and detailed description.

Any combination or permutation of embodiments is envisioned. Additional advantageous features, functions and applications of the disclosed processes and methods of the present disclosure will be apparent from the description which follows, particularly when read in conjunction with the appended figures.

### BRIEF DESCRIPTION OF THE DRAWINGS

The following figures are exemplary embodiments wherein the like elements are numbered alike.

Exemplary embodiments of the present disclosure are further described with reference to the appended figures. It is to be noted that the various features, steps, and combinations of features/steps described below can be arranged and organized differently to result in embodiments which are still within the scope of the present disclosure. To assist those of ordinary skill in the art in making, using, and practicing the disclosed processes and methods, reference is made to the appended figures, wherein:

The following figures are exemplary embodiments.

FIG. 1 is a schematic drawing of a process including an adiabatic (Houdry type) dehydrogenation unit.

FIG. 2A is a graph of daily isobutene produced/isobutane fed (wt./wt.) (“isobutene/isobutane”) versus Days on Stream (DOS) for historical data for a first cycle (Run 1), historical data for a second cycle (Run 2) and simulated data for both cycles with a “single polynomial” model.

FIG. 2B is a graph of isobutene/isobutane versus DOS for historical data for a first cycle (Run 1), historical data for a second cycle (Run 2) and simulated data for both cycles with a “two fragment linear” model.

FIG. 3A is a graph of methyl tert-butyl ether (MTBE) production (kilotons per annum (kTA)) versus Days on Stream (DOS) for the first cycle (Run 1).

FIG. 3B is a graph of US million dollars per annum (MMUSD/A) versus DOS for the first cycle (Run 1).

FIG. 4 is a schematic showing a strategy for maximizing annual profits from product sales.

FIG. 5A is a graph of daily isobutene/isobutane (wt./wt.) versus DOS for two runs of the examples.

FIG. 5B is a graph of isobutane-containing stream feed rate to the dehydrogenation reactor(s) (tons (T)/day) versus DOS for two runs of the examples.

FIG. 6A is a graph of cumulative isobutane fed (kilotons (kT)) versus DOS for two runs of the examples.

FIG. 6B is a graph of cumulative n-butane fed (kT) versus DOS for two runs of the examples.

FIG. 6C is a graph of cumulative 1,3-butadiene fed (kT) versus DOS for two runs of the examples.

FIG. 6D is a graph of cumulative olefins fed (kT) versus DOS for two runs of the examples.

FIG. 7A is a graph of cumulative n-butane fed (kT) versus cumulative feed (kT) for two runs of the examples.

FIG. 7B is a graph of cumulative 1,3-butadiene fed (kT) versus cumulative feed (kT) for two runs of the examples.

FIG. 7C is a graph of cumulative olefins fed (kT) versus cumulative feed (kT) for two runs of the examples.

FIG. 7D is a graph of cumulative (olefins−1,3-butadiene) fed (kT) versus cumulative feed (kT) for two runs of the examples.

FIG. 8A is a graph of 1,3-butadiene fed/n-butane fed (wt./wt.) versus DOS for two runs of the examples.

FIG. 8B is a graph of cumulative (1,3-butadiene/n-butane)×i-C4 (kT) versus DOS for two runs of the examples.

FIG. 9, which includes FIG. 5A and FIG. 8B, reports a graph of daily isobutene produced/isobutane fed (wt./wt.) versus DOS for two runs of the examples on the left y-axis, and the trend of cumulative (1,3-butadiene/n-butane)×i-C4 (kT) versus DOS for two runs of the examples on the right y-axis.

FIG. 10A is a graph of daily 1,3-butadiene produced/n-butane fed (wt./wt.) versus DOS for two runs of the examples.

FIG. 10B is a graph of daily (1,3-butadiene/n-butane)×i-C4 (kT) versus DOS for two runs of the examples.

FIG. 11A is a graph of daily isobutene produced/isobutane fed versus DOS for a third run of the examples, based on available plant data.

FIG. 11B is a graph of cumulative (1,3-butadiene/n-butane)×i-C4 (kT) versus DOS for a third run of the examples, based on available plant data.

FIG. 12 is a graph of cumulative (1,3-butadiene/n-butane)×i-C4 (kT) versus DOS for a third run based on available plant data and modeled/predicted trend for the remaining part of third run of the examples.

FIG. 13 is a graph of daily (1,3-butadiene/n-butane)×i-C4 (kT) versus DOS for a third run of the examples, based on available plant data.

FIG. 14 is a graph of daily isobutene produced/isobutane fed (wt./wt.) versus DOS for a model of the examples and actual data.

### DETAILED DESCRIPTION

The exemplary embodiments disclosed herein are illustrative of advantageous processes for operating a chemical process. It should be understood, however, that the disclosed embodiments are merely exemplary of the present disclosure, which may be embodied in various forms. Therefore, details disclosed herein with reference to exemplary processes and methods are not to be interpreted as limiting, but merely as the basis for teaching one skilled in the art the advantageous processes and methods of the present disclosure.

The present inventors have surprisingly found that prediction of the drop of performance over time on a stream of an alkane dehydrogenation unit based on the adopted operating philosophy may enable strategies aimed at optimizing run duration (with run duration defined as the time the unit is running between two turnarounds and dehydrogenation catalyst changeover), which may lead to a maximization of annual profit. The method may be capable of predicting process performance and drop of performance over time. The method relies on a set of equations defining unit performance from the start of the run towards the end of the run, which may enable the estimation of initial performance and drop of performance against process parameters and key performance/deactivation indicators. The method may be embedded in other tools/methodologies to provide a recommendation also based on economic Key Performance Indicators (KPIs).

Accordingly, disclosed herein is a process for operating a chemical process including deriving coefficients for a process performance model from historical feed data and historical production data; formulating the process performance model using the coefficients; determining a predicted change in production of a product of the chemical process using the process performance model; and changing a processing parameter of the chemical process based on economic data and the predicted change in production of the product of the chemical process.

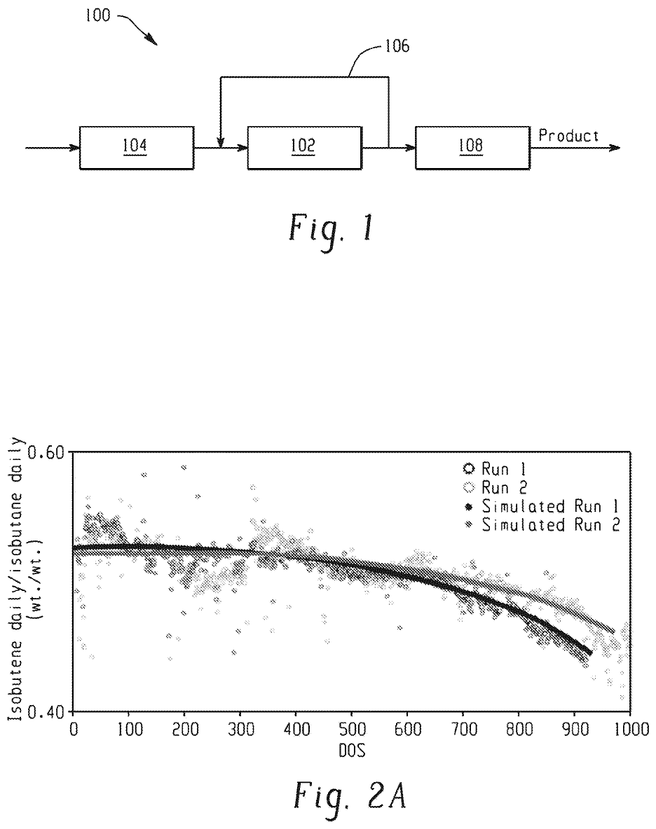

In an embodiment, the method described herein uses the analysis and quantification of chemical species present in the feed of a Houdry (adiabatic) type dehydrogenation process to quantify performance drop. FIG. 1 shows that the feed to a dehydrogenation unit 102 in a dehydrogenation process 100 may include the molecule desired to be dehydrogenated (e.g., isobutane and propane) and other molecules provided by both upstream unit(s) 104 and a recycle stream 106 from a downstream unit 108.

Impurities in a feed to the dehydrogenation unit 102 may be provided from an upstream physical separation process and the recycle stream 106. Process operation varies, and an amount of impurities entering the dehydrogenation unit may be case sensitive. The present inventors surprisingly found that an amount of specific molecules present in the feed to be dehydrogenated may be used as KPIs to assess performance and performance drop of the dehydrogenation unit. The present inventors surprisingly found for the isobutane dehydrogenation process that performance drop may be correlated, for example, with the amount of olefins, 1-butene, 2-butene, 1,3-butadiene, n-butane and isobutane (also referred to herein as i-C4) fed into the unit. The present inventors also surprisingly found that certain mathematical equations relating the parameters disclosed herein could be used in a model which accurately predicted a drop of performance of the dehydrogenation unit.

For an alkane, e.g., isobutane, dehydrogenation reaction, the present inventors surprisingly found that the isobutene to isobutane ratio at a certain time t, could be predicted by linear or polynomial functions, covering the intended duration of a run defined. In an embodiment, a single or multiple polynomial interpolation/prediction may be used, and the following equations may be used to calculate the isobutene/isobutane ratio at a certain time (Equation (1)) and cumulative isobutene produced (Equation (2)):

$$\frac{daily i = {C}_{4 PROD}}{daily i - {C}_{4 FED}} ( t ) = \frac{daily i = {C}_{4 PROD}}{daily i - {C}_{4 FED}} ( {t}_{0} ) - {m}_{1} \cdot {t}^{2} ( DOS ) + {m}_{2} \cdot t ( DOS ) \qquad ( 1 )$$

$$Cumulative isobutene = \qquad ( 2 )$$

$${\sum }_{0}^{{t}_{1}} ( {F}_{i} \cdot \frac{{M}_{[ isobutene ]}}{{M}_{[ isobutane ]}} ) + {\sum }_{{t}_{1}}^{{t}_{2}} ( {F}_{( i + 1 )} \cdot \frac{{M}_{[ isobutene ]}}{{M}_{[ isobutane ]}} ) + …$$

In Equations (1)-(7), i=C_{4PROD} is the amount of isobutene produced, t is a certain time, i-C_{4FED} is the amount of isobutane fed to a dehydrogenation unit, m, m₁, and m₂ are coefficients, F is averaged daily flow rate to be multiplied by i=C_{4PROD}/i-C_{4FED} at t, DOS is Days On Stream, M is daily mass, and Wᵢ, Wᵢᵢ, Wᵢᵢᵢ, Wᵢᵢᵢᵢ, and Wᵢᵢᵢᵢᵢ vary between −1,000 and 1,000.

In an embodiment, a single or multiple linear interpolation/prediction may be used, and the following equations may be used to calculate the isobutene/isobutane ratio at a certain time (Equation (3)) and cumulative isobutene produced (Equation (2)):

$$\frac{daily i = {C}_{4 PROD}}{daily i - {C}_{4 FED}} ( t ) = \frac{daily i = {C}_{4 PROD}}{daily i - {C}_{4 FED}} ( {t}_{0} ) - m \cdot t ( DOS ) \qquad ( 3 )$$

wherein m is a slope coefficient, as defined herein.

In an embodiment, the timespan within which a certain set of equations and related coefficients m, m₁, and m₂ (Equations (1)-(3)) may be used to predict unit performance, N, may be calculated by Equation (4):

$$N = ( {\sum }_{0}^{t} {M}_{[ Specie A ]} ) \cdot {W}_{i} + ( {\sum }_{0}^{t} \frac{{M}_{[ Specie A ]}}{{M}_{[ Feed ]}} ) \cdot {W}_{ii} + ( {\sum }_{0}^{t} \frac{{M}_{[ Specie A ]}}{{M}_{[ Specie B ]}} ) \cdot {W}_{iii} + ( {{\sum }_{0}}^{t} \frac{{M}_{[ Specie A ]} \cdot {M}_{[ Specie C ]}}{{M}_{[ Feed ]}} ) \cdot {W}_{iiii} + ( {\sum }_{0}^{t} \frac{{M}_{[ Specie A ]} \cdot {M}_{[ Specie C ]}}{{M}_{[ Specie B ]}} ) \cdot {W}_{iiiii} \qquad ( 4 )$$

Even if all terms in Equation (4) are summed up, this may not be limitative as some of the terms may be subtracted or multiplied as well. The correct time to use a different equation set and m, m₁, and m₂ coefficients may be determined when the numeric value of N reaches a certain range. In Equation (4), the term M stands for the daily mass of a certain component or components mixture and the coefficients Wᵢ, Wᵢᵢ, Wᵢᵢᵢ, Wᵢᵢᵢᵢ, and Wᵢᵢᵢᵢᵢ might vary between −1,000 and 1,000.

In an embodiment, the disclosed process addresses, e.g., may be directed to, a plant comprising a catalytic dehydrogenation unit processing light alkanes, for example, a plant comprising a “Houdry” type dehydrogenation unit processing isobutane, propane or a combination of both, or a plant producing MTBE and comprising a “Houdry” type dehydrogenation unit processing isobutane.

Houdry or adiabatic dehydrogenation units for isobutane, propane or mixed C₃/C₄ feed dehydrogenation may include multiple (e.g., three or more) reactors operated in parallel, and a new value N′, e.g., an updated timespan, defined by Equation (5) is defined to take into account for different reactor configuration and provide “normalized” results.

$${N}^{′} = \frac{N}{{M}_{Active Material}} \cdot X + \frac{N}{{M}_{Inert}} \cdot Y + \frac{N}{( {M}_{Active material} + {M}_{Inert} )} \cdot Z . \qquad ( 5 )$$

In Equation (5), “M_{Active Material}” represents the mass of material playing an active role in either transforming alkane molecules to olefins, playing an active role in the heat balance of the dehydrogenation reactors, or a combination thereof. “M_{Inert}” represents the mass of material not playing an active role in chemical transformation, heat balance of the dehydrogenation reactors, or a combination thereof. N is the timespan within which the certain set of equations and related coefficients are used to predict unit performance calculated using Equation (4). X, Y and Z are coefficients and may vary between 0 and 1. The N′ coefficient may be used optionally for units having different reactor configurations and the decision to use a different equation set and the m coefficient (Equation (3)) and the m₁ and m₂ coefficients (Equation (1)) may be determined when the numeric value of N′ reaches a certain range.

The coefficients used to determine the predicted drop of performance with both the polynomial and the linear approaches (e.g., the mᵢ coefficients) may be based on the same KPIs used to define the timespan within which a certain set of equations may be used. In an embodiment, the mᵢ coefficients may be calculated by a derivative approach as shown in Equation (6):

$${M}_{i} = {W}_{i} \cdot \frac{d}{dt} {M}_{[ Specie A ]} + {W}_{ii} \cdot \frac{d}{dt} ( \frac{{M}_{[ Specie A ]}}{{M}_{[ Feed ]}} ) + {W}_{iii} \cdot \frac{d}{dt} ( \frac{{M}_{[ Specie A ]}}{{M}_{[ Specie B ]}} ) + {W}_{iiii} \cdot \frac{d}{dt} ( \frac{{M}_{[ Specie A ]} \cdot {M}_{[ Specie C ]}}{{M}_{[ Feed ]}} ) + {W}_{iiiii} \cdot \frac{d}{dt} ( \frac{{M}_{[ Specie A ]} \cdot {M}_{[ Specie C ]}}{{M}_{[ Specie B ]}} ) \qquad ( 6 )$$

Coefficients Wᵢ, Wᵢᵢ, Wᵢᵢᵢ, Wᵢᵢᵢᵢ, and Wᵢᵢᵢᵢᵢ used in Equation (6) may be equal to or different than those used in Equation (4). Coefficients used in Equations (4) and (6) may be unit sensitive, e.g., specific coefficients may apply for a targeted dehydrogenation unit. The coefficient(s) to be used in Equations (4) and (6) may be obtained from historical unit data analysis and correlation. In an embodiment, the chemical process includes alkane dehydrogenation, and the coefficients may include m (Equation (3)); m₁ and m₂ (Equation (1)); and Mᵢ, Wᵢ, Wᵢᵢ, Wᵢᵢᵢ, Wᵢᵢᵢᵢ, and Wᵢᵢᵢᵢᵢ (Equation (6)).

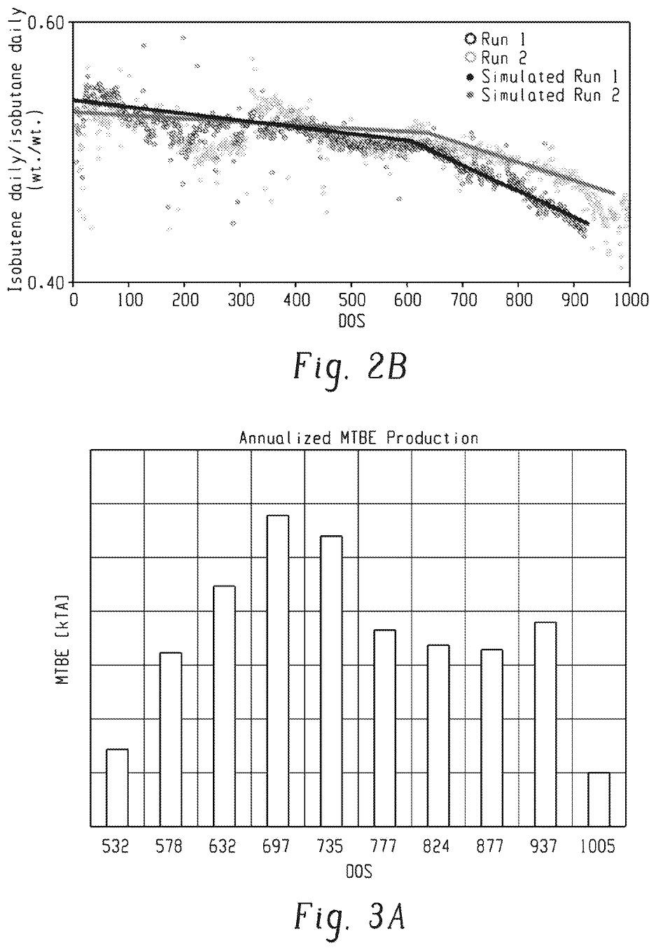

FIG. 2A shows use of the equations and coefficients disclosed herein to predict isobutene/isobutane ratio with a “single polynomial” approach for two Runs (i.e. Run 1 and Run 2) carried out in an isobutane dehydrogenation commercial unit, and FIG. 2B shows use of the equations and coefficients disclosed herein to predict isobutene/isobutane ratio with a “two fragments linear” approach for two Runs (i.e. Run 1 and Run 2) carried out in the isobutane dehydrogenation commercial unit.

The number of fragments that may be used to achieve good prediction of plant performance may have no limit in number. In an embodiment, good prediction may be achieved with a number of linear or polynomial fragments ranging from 1 to 20, for example, from 1 to 5.

Model prediction accuracy for a certain run may increase progressively during run execution. In an embodiment, the model may be run at least on a monthly basis.

The model may be used alone or in combination with other catalyst kinetic/deactivation based models. The disclosed olefins may not be the final product, and losses that may occur in downstream units may also be considered to predict the final product production over time. The disclosed process may be embedded in any suitable calculation or model describing, e.g., involving, upstream and downstream units. The disclosed model may be run monthly, and the calculation or model describing, e.g., involving, upstream and downstream units may be run with the same frequency to improve prediction accuracy with respect to the final product.

Once final product production forecast over time on stream is achieved, the results may be used to define the “optimal run duration” with the aim of maximizing annual profit, which may be carried out by including in the calculation economic KPIs, for example, contribution margin from product sales, earnings before interest, taxes, depreciation, and amortization (EBITDA), maintenance cost (including turnaround cost), catalyst cost, or a combination thereof.

Accordingly, the economic data of the disclosed process may include contribution margin from sales of the product of the chemical process; earnings before interest, taxes, depreciation, and amortization; maintenance cost; catalyst cost; or a combination thereof. In an embodiment, changing the processing parameter of the disclosed process may include changing the catalyst and catalyst bed configuration. The process may further include updating the process performance model. Updating the process performance model may include using additional feed data and additional production data. In an embodiment, updating the process performance model includes using updated economic data, additional economic data, or a combination thereof.

An example showing how annualized MTBE production and annual profit changes in relation to run (cycle) duration using the combined disclosed methods is shown in FIG. 3A and FIG. 3B. In the case of a plot reporting annualized profit from MTBE sales, results may be given for a two year (24 months) or three year (36 months) run (or cycle) duration reference, and delta (positive or negative) for other run duration may be reported with respect to this reference. Annualized production may be reported as absolute values. The peak of annualized production may not correspond to the maximized yearly profit and economic KPIs may affect the assessment of the “ideal run duration.” In the two plots, annualized MTBE production and annualized yearly profit is simulated for run or cycles having a duration in the 17-33 Months On Stream (MOS) range, which may be the timeframe MTBE plants are run between turnarounds and catalyst changeover. In an embodiment, run duration outside a 17-33 MOS range window may be successfully simulated.

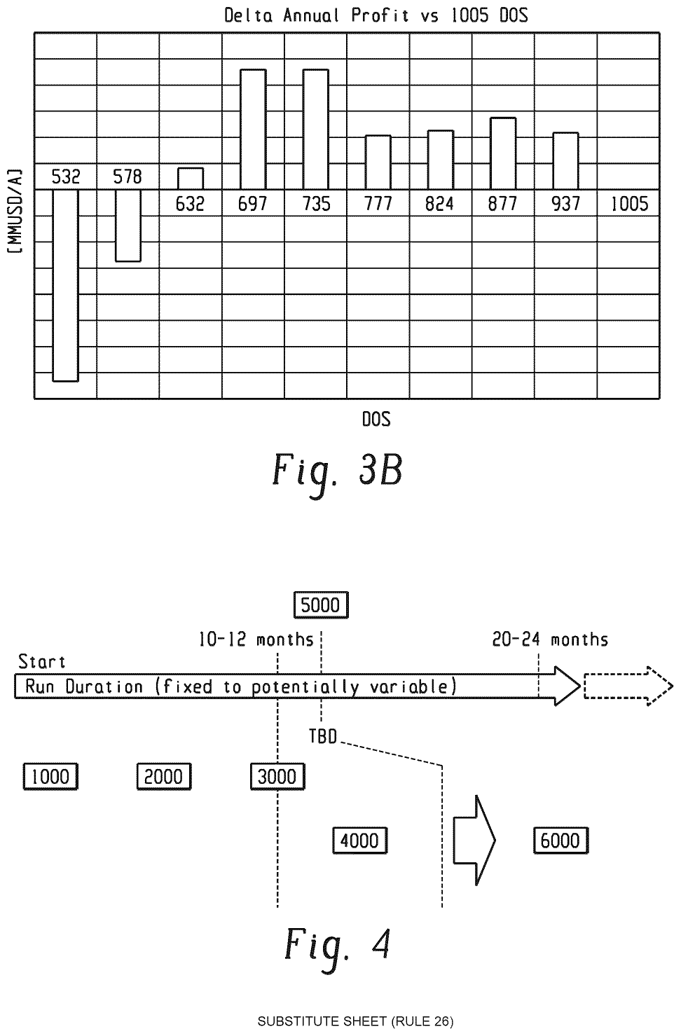

FIG. 4 shows a strategy that may be used for the successful implementation of the disclosed tool. At a “pre-start” phase 1000, the model may be available with precompiled information referring to a “standard” case for a selected train, e.g., reactor(s) or plant; the model will allow for change of the precompiled information (e.g., preloaded parameters) according to a business plan and forecasted economic and other technical factors; based on the precompiled information (e.g., preloaded parameters), the model will suggest operating conditions to maximize production for a given cycle duration, or a combination thereof.

During a “from start to catalyst procurement” phase 2000, the model may be updated monthly by utilizing real production and operating data; the model may be updated monthly by utilizing real economic data; technical data may be used by the model to predict dehydrogenation drop of performance based on operating conditions; or a combination thereof. A “catalyst procurement” phase 3000 may occur 10-12 from the “pre-start” phase 1000, and under the assumption that the catalyst lead time is 10 months.

During a “from catalyst procurement to point of decision” phase 4000, the model may be updated bi-weekly or monthly by utilizing real production and operating data; the model may be updated monthly by utilizing real economic data; in case of unattended events (e.g., unplanned shutdown), the model may be updated promptly; technical data may be used by the model to predict dehydrogenation drop of performance based on operating conditions; or a combination thereof. An “ultimate decision on cycle duration” 5000, may depend on, for example, turnaround planning activities requirements in subsidiaries, notice time due to contract, or a combination thereof.

During a “Model Prediction for Optimal Cycle duration” phase 6000, there may be an evaluation of difference(s) between initial and updated assessments; a decision on cycle duration based on identified gap and additional key factors; or a combination thereof.

Determining a predicted change in production of the product of the chemical process using the process performance model may include determining more than one predicted change in production of the product of the chemical process using the process performance model.

In an embodiment, the model can be used in combination with or be embedded in software tools such as, for example, plant DCS (Distributed Control System), PIMS (Plant Information Management System), PI System (e.g., application software for real-time data management), LIMS (Laboratory Information Management System), SAP (e.g., systems, applications, and products), or a combination thereof.

This disclosure is further illustrated by the following examples, which are non-limiting.

### Examples

### Development of the Prediction Model Based on a Two Segments Linear Approach

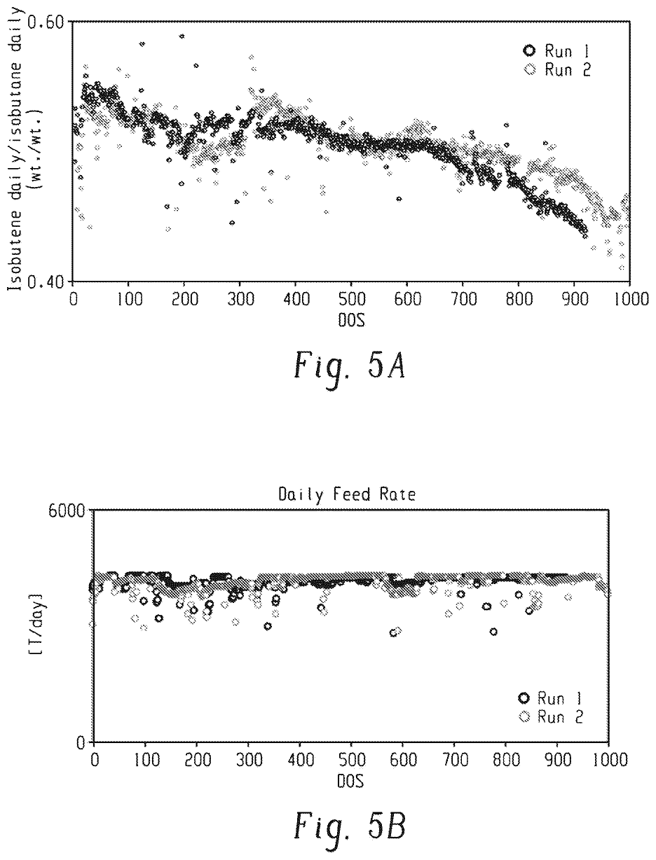

The model coefficients Wᵢ were obtained by studying, analyzing and interpolating historical plant data. FIG. 5A is a graph of isobutene/isobutane versus DOS for two different runs processed by a dehydrogenation unit and FIG. 5B is a graph of isobutane-containing stream feed rate (tons (T)/day) versus DOS for the two different runs processed by the dehydrogenation unit.

Although the feed rate was similar in Run 1 and Run 2, performance drop (trends (e.g., slopes) of produced isobutene/isobutane versus DOS) differed especially in the last period of the runs (i.e., from about 650 DOS) with Run 1 showing an earlier and more severe performance drop in comparison with Run 2. Trends and evolution of species present in the feed sent to the dehydrogenation unit were studied, initially by taking into consideration single species, categories, or a combination thereof.

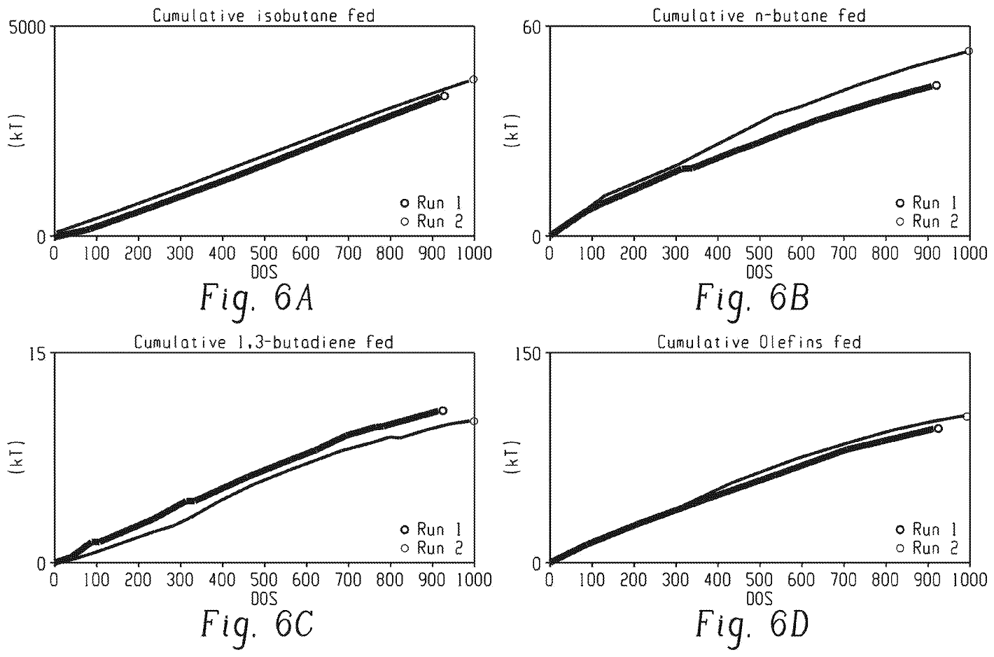

FIG. 6A, FIG. 6B, FIG. 6C, and FIG. 6D show the single species or categories (e.g., olefins) used to derive Wᵢ coefficients. Each graph represents graphically the terms (Σ₀^{t} M_{[Specia A]}) in Equation (4):

$$N = ( {\sum }_{0}^{t} {M}_{[ Specie A ]} ) \cdot {W}_{i} + ( {\sum }_{0}^{t} \frac{{M}_{[ Specie A ]}}{{M}_{[ Feed ]}} ) \cdot {W}_{ii} + ( {\sum }_{0}^{t} \frac{{M}_{[ Specie A ]}}{{M}_{[ Specie B ]}} ) \cdot {W}_{iii} + ( {\sum }_{0}^{t} \frac{{M}_{[ Specie A ]} \cdot {M}_{[ Specie C ]}}{{M}_{[ Feed ]}} ) \cdot {W}_{iiii} + ( {\sum }_{0}^{t} \frac{{M}_{[ Specie A ]} \cdot {M}_{[ Specie C ]}}{{M}_{[ Specie B ]}} ) \cdot {W}_{iiiii} \qquad ( 4 )$$

wherein:
Specie A includes: propane, propene, propadiene, iso-butane, n-butane, iso-butene 1-butene, 2-butene, 1,3-butadiene and olefins (sum of all species having one or more double bonds in the structure);
Specie B includes: propane, n-butane, iso-butane;
Specie C includes: iso-butane, propane; and
Wᵢ, Wᵢᵢ, Wᵢᵢᵢ, Wᵢᵢᵢᵢ, Wᵢᵢᵢᵢᵢ are numeric coefficients by which each term of the equation is multiplied.

Thus, in the non-limitative case of this example, the general term (Σ₀^{t} M_{[specie A]})·Wᵢ in Equation (4) will be given by (Σ₀^{t} M_{[iso-butane]})·W₁+(Σ₀^{t} M_{[n-butane]})·W₂+(Σ₀^{t} M_{[1,3-butadiene]})·W₃+(Σ₀^{t} M_{[olefins]})·W₄.

Some of above terms are not proportional to each other. For example, the numerical value of term (Σ₀^{t} M_{[1,3-butadiene]}) is lower for Run 2 in comparison with Run 1, while the terms (Σ₀^{t} M_{[olefins]}) and (Σ₀^{t} M_{[n-butane]}) are higher for Run 2 than Run 1. Thus, while (Σ₀^{t} M_{[specie A]}) for isobutane, n-butane, and olefins are higher for Run 2 than Run 1, this is not the case for 1,3-butadiene, which also contributes to the term (Σ₀^{t} M_{[olefins]).}

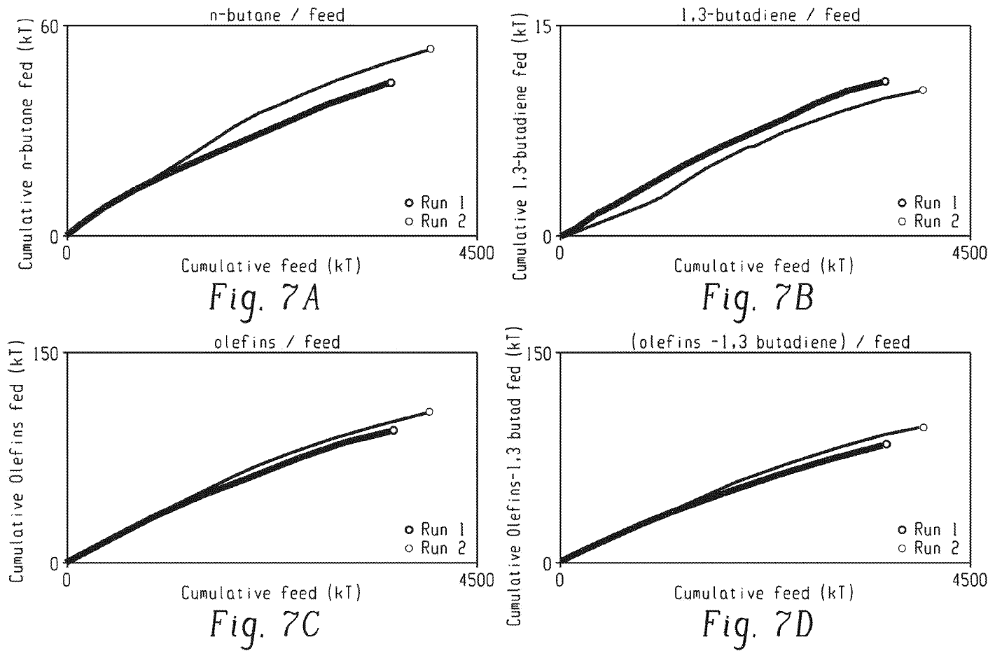

Trends were also studied when normalized with respect to cumulative feed or cumulative isobutane sent to the dehydrogenation unit. Trends normalized with respect to cumulative feed are shown in FIG. 7A, FIG. 7B, FIG. 7C, and FIG. 7D. Each plot represents graphically the terms (Σ₀^{t} M_{[specie A]}/M_{[Feed]}) in Equation (4). Thus, in the non-limitative case of this example, the general term (Σ₀^{t} M_{[specie A]}/M_{[Feed]})·Wᵢᵢ in Equation (4) will be given by (Σ₀^{t} M_{[n-butane]}/M_{[Feed]})·W₅+(Σ₀^{t} M_{[1,3-butadiene]}/M_{[Feed]})·W₆+(Σ₀^{t} M_{[olefins]}/M_{[Feed]})·W₇+(Σ₀^{t} M_{[olefins-1,3-butadiene]}/M_{[Feed]})·W₈.

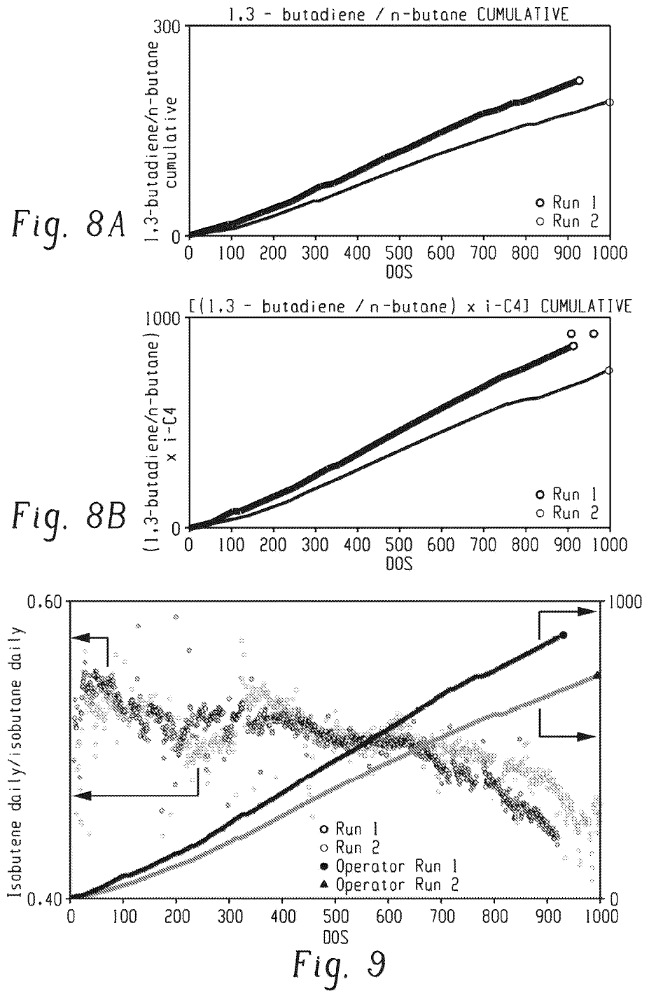

Historical data were analyzed and trends studied as shown in FIG. 8A and FIG. 8B. “Operators” (e.g., terms of Equation (4)) obtained by mathematical processing of certain Key Performance Indicators (KPIs) were derived. FIG. 8A provides values for the general term (Σ₀^{t} M_{[specie A]}/M_{[Specie B]}). Thus, in the non-limitative case of this example, the general term (Σ₀^{t} M_{[specie A]}/M_{[Specie B]})·Wᵢᵢᵢ in Equation (4) will be given by (Σ₀^{t} M_{[1,3-butadiene]}/M_{[n-butane]})·W₉. FIG. 8B provides values for the general term (Σ₀^{t} M_{[specie A]}·M_{[Specie C]}/M_{[Specie B]}) of Equation (4) and more specifically, in the non-limitative case of this example, the general term (Σ₀^{t} M_{[specie A]}·M_{[Specie C]}/M_{[Specie B]})·Wᵢᵢᵢᵢᵢ of Equation (4) will be given by (Σ₀^{t}[M_{[1,3-butadiene]}·M_{[iso-butane]}/M_{[n-butane]]})·W₁₀.

Referring to FIG. 6A, FIG. 6B, FIG. 6C, FIG. 6D, FIG. 7A, FIG. 7B, FIG. 7C, FIG. 7D, FIG. 8A, and FIG. 8B, trends and terms in Equation (4) differ for the two runs studied, and the time at which a different deactivation slope should be used can be identified. The point at which a new deactivation slope shall be utilized is given by the term “N” in Equation (4). In particular, when “N” reaches a certain value or a certain value range a new slope will allow a better prediction of loss of performance. Moreover, since values for Wᵢ, Wᵢᵢ, Wᵢᵢᵢ, Wᵢᵢᵢᵢ, and Wᵢᵢᵢᵢᵢ can change between −1,000 and 1,000, each individual term of Equation (4) could have more or less weight in determining the value of the term “N”.

FIG. 9 shows a non-limitative example where Wᵢ, Wᵢᵢ, Wᵢᵢᵢ and Wᵢᵢᵢᵢ coefficients are set to zero and the term “N” in Equation (5) is only determined by the term “Σ₀^{t}(M_{[1,3-butadiene]}·M_{[i-C4]}/M_{[n-butane]})” with Wᵢᵢᵢᵢᵢ equal to 1. Thus, in this case, change from “single polynomial” to “two fragment linear,” is only determined by the term “Σ₀^{t}(M_{[1,3-butadiene]}·M_{[i-C4]}/M_{[n-butane]})” of Equation (4).

FIG. 9 shows that when the term “Σ₀^{t}(M_{[1,3-butadiene]}·M_{[i-C4]}/M_{[n-butane]})” reaches a certain value a slope of daily isobutene/isobutane changes and can be well fitted with two different linear interpolations: a first to be used when the term “Σ₀^{t}(M_{[1,3-butadiene]}·M_{[i-C4]}/M_{[n-butane]})” is less than 500 to 650 kT and a second to be used when the term “Σ₀^{t}(M_{[1,3-butadiene]}·M_{[i-C4]}/M_{[n-butane]})” is greater than 500 to 650 kT.

After the DOS or time range at which deactivation slope change is determined, the slope of deactivation of each segment can be determined. The slope can be determined by interpolating plant data for daily isobutene/isobutane.

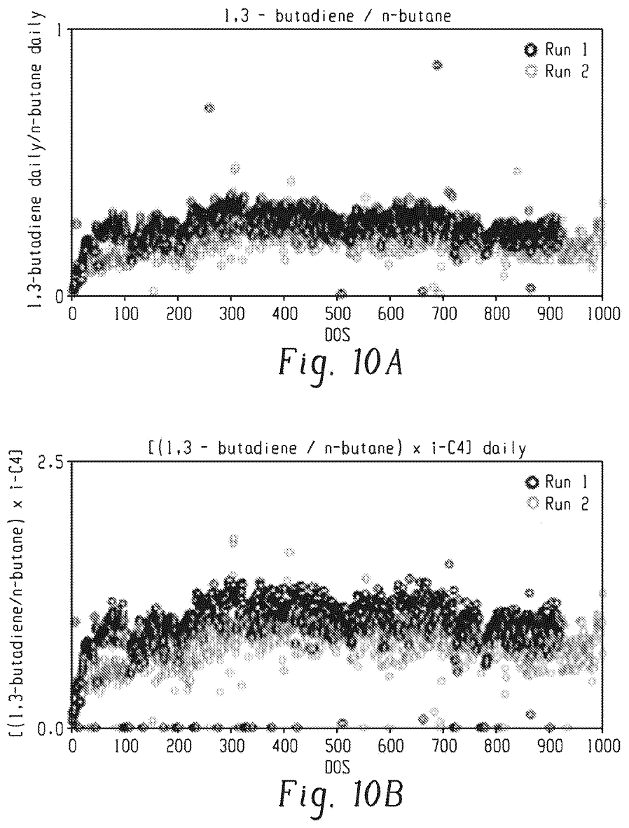

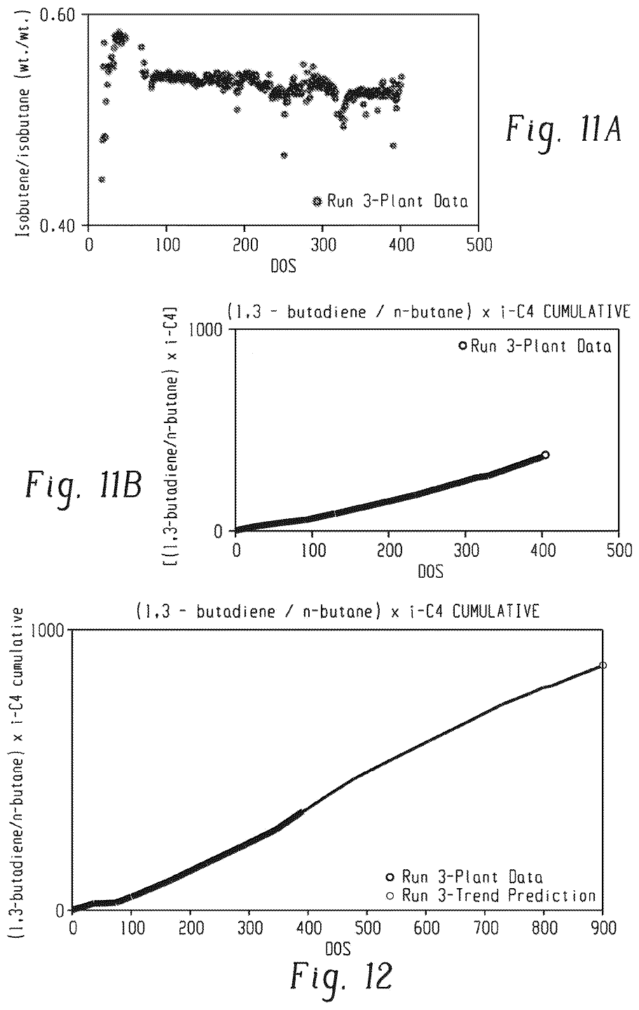

The calculated slopes could be linked to the same operator(s) but in a derivative form, as reported in Equation (6). Species A, B and C in Equation (6) are defined as reported for Equation (4). FIG. 10A shows a non-limitative example where Wᵢ, Wᵢᵢ, Wᵢᵢᵢ, and Wᵢᵢᵢᵢᵢ are equal to zero, while Wᵢᵢᵢᵢ is equal to 1 and the term (1,3-butadiene/n-butane) is used. FIG. 10B shows another non-limitative example where Wᵢ, Wᵢᵢ, Wᵢᵢᵢ, and Wᵢᵢᵢᵢ are equal to zero, while Wᵢᵢᵢᵢᵢ is equal to 1 and the term (1,3-butadiene/n-butane)×i-C4 is used.

Each of FIG. 10A and FIG. 10B can be divided in the following sections: a first time period of the run, in which operator daily trends increase (d/dt>0). In the first time period of the run, catalyst is fresh and active and at the same time heat input to the reactor is increased progressively to sustain isobutane yield. After about 300-400 DOS operator(s) daily trends change reaching a plateau (d/dt=0), followed by decrease (d/dt<0), which can be explained considering that at a certain DOS heat input to the reactors is maximized and cannot be further increased while catalyst deactivates with time. Slope of the operators in the time periods of the run in which d/dt are either positive or negative can be used to link Equation (6) with the deactivation slopes of the two segments derived from FIG. 2B.

### Example Proving the Validity of the Approach in Predicting Plant Performance

After the model was developed, it was validated in order to assess the prediction capabilities thereof using an ongoing run on the same plant using data available for the first 400 DOS. FIG. 11A shows isobutene/isobutane (wt./wt.) in the reactor product and FIG. 11B shows the cumulative trend of the term “Σ₀^{t}(M_{[1,3-butadiene]}·M_{[i-C4]}/M_{[n-butane]})” with Wᵢᵢᵢᵢᵢ coefficient equal to 1 for the first 400 DOS.

Within the first 400 DOS, dehydrogenation performance could be described by a “single” linear interpolation which is in agreement with the fact that the term “Σ₀^{t}(M_{[1,3-butadiene]}·M_{[i-C4]}/M_{[n-butane]})” was still less than 500 to 650 kT.

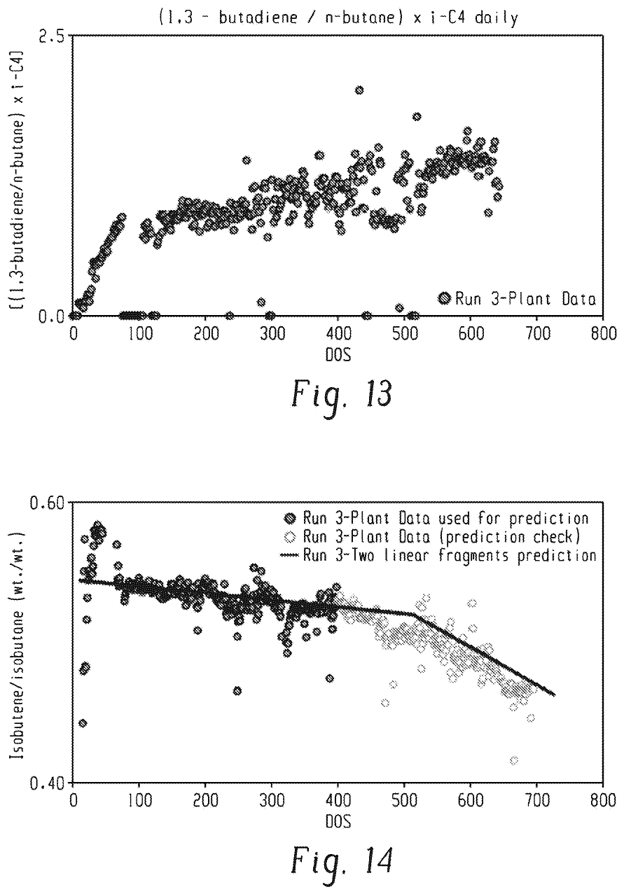

Successively, these data were used to predict future trends and values of the term “Σ₀^{t}(M_{[1,3-butadiene]}·M_{[i-C4]}/M_{[n-butane]})” in order to assess after how many DOS the second linear interpolation with higher slope should have been used to better predict evolution of isobutene/isobutane for a specified feed rate, as shown in FIG. 12. Referring to FIG. 12, the slope of isobutene/isobutane was expected to change after 500-550 DOS. Next, estimation of the slope coefficient “m” for the second linear interpolation was carried out, using the term (1,3-butadiene/n-butane)×i-C4 and assuming that Wᵢᵢᵢᵢᵢ coefficient in Equation (6) for the term (1,3-butadiene/n-butane)×i-C4 is equal to 1 while the Wᵢ, Wᵢᵢ, Wᵢᵢᵢ, and Wᵢᵢᵢᵢ coefficients are equal to zero. Trends for the term (1,3-butadiene/n-butane)×i-C4 in the first 400 DOS of Run 3 are shown in FIG. 13.

The daily trend of the term (1,3-butadiene/n-butane)×i-C4 was similar to the trend shown in FIG. 10 for Run 1 and Run 2 (only d/dt trends for the first 300-400 DOS changes) allowing the calculation of the deactivation slope “m” to be used for the second linear interpolation. Results of the assessment and performance prediction under the assumption of a specified and constant feed rate to the dehydrogenation reactors are shown in FIG. 14 and compared with plant data obtained after the prediction was delivered.

The model successfully predicted both (a) at which time the second deactivation slope should be used and (b) the correct slope for isobutene/isobutane drop over time in the second segment of linear interpolation. Based on interpolation and prediction shown in FIG. 12, change in slope was estimated to happen after about 500-550 DOS and with a slope for the second linear fragment similar to the slope that occurred in the plant thereafter.

Dehydrogenation technology is influenced by heat balance in the reactors, and isobutene/isobutane versus time (slope) might differ from prediction if feed rate is decreased (e.g., decreased by greater than 2%), especially when a change in operation is carried out within the second part of the run (i.e., in the second linear fragment). The “m” coefficient of the second linear fragment might be overestimated. In fact, isobutene/isobutane is a complex function of reactor(s) heat balance, which is normally determined by feed rate and heat inputs to the reactors. This method predicts deactivation slope(s) during the second part of the run under the assumption of a constant heat input/feed ratio. Thus, if for instance feed rate is decreased significantly, predicted slope is overestimated since actual heat input/feed ratio is higher than the one existing while estimating performance drop. However, although isobutene/isobutane might be higher than expected and consequently slope lower, the impact on overall isobutene production drop might be buffered as the feed rate is lower than the one used during prediction as reported in Equation (2).

The results of the disclosed methodology can be further processed in order to predict final product (e.g., methyl tert-butyl ether (MTBE)) annualized production and “ideal run duration” with the aim of maximizing annual profit.

The coefficients Wᵢ, Wᵢᵢ, Wᵢᵢᵢ, Wᵢᵢᵢᵢ, Wᵢᵢᵢᵢᵢ may be calculated in accordance with the following. In an embodiment, for 500 DOS, coefficients can be calculated according to the procedure disclosed herein for Run 1 and Run 2.

TABLE 1

| COLUMN | A | B | C | D | E |
|---|---|---|---|---|---|
| Coefficient for | Run 1 | Run 2 | Run 1-Run 2 | Delta % | Normalization |
| (Σ₀^{t} M_{[iso-butane]}) | 1756.985 | 1828.461 | −71.47690 | −3.99 | −0.093 |
| (Σ₀^{t} M_{[n-butane]}) | 26.429 | 32.228 | −5.79875 | −19.77 | −0.459 |
| (Σ₀^{t} M_{[1,3-butadiene]}) | 6.649 | 6.063 | 0.58659 | 9.23 | 0.214 |
| (Σ₀^{t} M_{[olefins]}) | 59.062 | 64.582 | −5.51989 | −8.93 | −0.207 |
| Σ₀^{t} M_{[n-butane]}/M_{[Feed]}) | 0.014 | 0.016 | −0.00225 | −14.77 | −0.343 |
| Σ₀^{t} M_{[1,3-butadiene]}/M_{[Feed]}) | 0.004 | 0.003 | 0.00047 | 14.25 | 0.331 |
| Σ₀^{t} M_{[olefins]}/M_{[Feed]}) | 0.031 | 0.033 | −0.00125 | −3.89 | −0.090 |
| Σ₀^{t} M_{[olefins-1,3-butadiene]} /M_{[Feed]}) | 0.028 | 0.030 | −0.00172 | −5.98 | −0.139 |
| (Σ₀^{t}[M_{[1,3-butadiene]}/M_{[n-butane]}]) | 121.458 | 96.595 | 24.86244 | 22.80 | 0.530 |
| (Σ₀^{t}[M_{[1,3-butadiene]} · M_{[iso-butane]}/M_{[n-butane]}]) | 462.013 | 365.725 | 96.28819 | 23.27 | 0.541 |

The value of a single species, as for instance (Σ₀^{t} M_{[iso-butane]}), is calculated at 500 DOS. The calculated values are reported in Table 1 in column A and B for Run 1 and Run 2 respectively. Analogously, the value of other descriptors are also reported at 500 DOS in column A and B for Run 1 and Run 2 respectively. The values reported in columns C, D and E are calculated from the values in column A and B as follows. Values in column C are the mathematical difference of values of column A and B (i.e., Run 1−Run 2). Values in column D (Delta %) are calculated by the following operation: C×100/[(A+B)/2]. Values in column E (Normalization) are calculated by considering the difference between the lower and the higher value amongst all the considered descriptors in column D. In the case given in this example, these are −19.77 and 23.27 which gives a difference of 43.04. The values in column E are thus calculated by the following operation: D/43.04.

Coefficients Wᵢ, Wᵢᵢ, Wᵢᵢᵢ, Wᵢᵢᵢᵢ, Wᵢᵢᵢᵢᵢ can be chosen either from column C, or D or E, given the boundary that once a column is chosen, only the values of that column shall be used as coefficients. In addition, a coefficient is set to zero in case a duplication of any of the descriptors of Equation (4) occurs. The same applies for Equation (6). This is for instance the case of descriptor (W M_{[specie A]}) and descriptor (Σ₀^{t} M_{[specie A]}·M_{[Specie C]}/M_{[Specie B]}) when Specie B and Specie C is isobutane. Accordingly, the coefficient used for descriptor (Σ₀^{t} M_{[specie A]}·M_{[Specie C]}/M_{[Specie B]}) is set to zero. In addition to the above, any of the coefficients multiplying one or more descriptors in Equation (4) or in Equation (6) can be arbitrarily set to zero regardless of the results reported in Table 1. This is the case, for instance, of the example given in FIG. 9. In this example, if one takes column E as a reference for calculating the coefficients, the following operation shall be considered. The lower value in column D is set to zero while the higher is set to 23.27 and thus 23.27-0=23.27, giving the coefficient multiplying descriptor “Σ₀^{t}(M_{[1,3-butadiene]}·M_{[i-C4]}/M_{[n-butane]})” equal to 1 since D/23.27=23.27/23.27=1.

This disclosure further encompasses the following aspects.

Aspect 1. A process for operating a chemical process comprising: deriving coefficients for a process performance model from historical feed data and historical production data; formulating the process performance model using the coefficients; determining a predicted change in production of a product of the chemical process using the process performance model; and changing a processing parameter of the chemical process based on economic data and the predicted change in production of the product of the chemical process.

Aspect 2. The process of Aspect 1, wherein the economic data comprises contribution margin from sales of the product of the chemical process; earnings before interest, taxes, depreciation, and amortization; maintenance cost; catalyst cost; or a combination thereof.

Aspect 3. The process of Aspect 1 or 2, wherein changing the processing parameter comprises changing the catalyst and catalyst bed configuration.

Aspect 4. The process of any of the preceding aspects, further comprising updating the process performance model.

Aspect 5. The process of Aspect 4, wherein updating the process performance model comprises using additional feed data and additional production data.

Aspect 6. The process of Aspect 4 or 5, wherein updating the process performance model comprises using updated economic data, additional economic data, or a combination thereof.

Aspect 7. The process of any of the preceding aspects, wherein the chemical process comprises alkane dehydrogenation.

Aspect 8. The process of Aspect 7, wherein the coefficients comprise m, m₁, m₂, Mᵢ, Wᵢ, Wᵢᵢ, Wᵢᵢᵢ, Wᵢᵢᵢᵢ, and Wᵢᵢᵢᵢᵢ in Equations (1), (3), and (6):

$$\frac{daily i = {C}_{4 PROD}}{daily i - {C}_{4 FED}} ( t ) = \frac{daily i = {C}_{4 PROD}}{daily i - {C}_{4 FED}} ( {t}_{0} ) - {m}_{1} \cdot {t}^{2} ( DOS ) + {m}_{2} \cdot t ( DOS ) \qquad ( 1 )$$

$$\frac{daily i = {C}_{4 PROD}}{daily i - {C}_{4 FED}} ( t ) = \frac{daily i = {C}_{4 PROD}}{daily i - {C}_{4 FED}} ( {t}_{0} ) - m \cdot t ( DOS ) \qquad ( 3 )$$

$${M}_{i} = {W}_{i} \cdot \frac{d}{dt} {M}_{[ Specie A ]} + {W}_{ii} \cdot \frac{d}{dt} ( \frac{{M}_{[ Specie A ]}}{{M}_{[ Feed ]}} ) + {W}_{iii} \cdot \frac{d}{dt} ( \frac{{M}_{[ Specie A ]}}{{M}_{[ Specie B ]}} ) + {W}_{iiii} \cdot \frac{d}{dt} ( \frac{{M}_{[ Specie A ]} \cdot {M}_{[ Specie C ]}}{{M}_{[ Feed ]}} ) + {W}_{iiiii} \cdot \frac{d}{dt} ( \frac{{M}_{[ Specie A ]} \cdot {M}_{[ Specie C ]}}{{M}_{[ Specie B ]}} ) \qquad ( 6 )$$

wherein
i=C_{4PROD} is amount of isobutene produced,
t is a certain time,
i-C_{4FED} is amount of isobutane fed to a dehydrogenation unit,
F is averaged daily flow rate to be multiplied by i=C_{4PROD}/i-C_{4FED} at t,
DOS is Days On Stream,
M is daily mass, and
Wᵢ, Wᵢᵢ, Wᵢᵢᵢ, Wᵢᵢᵢᵢ, and Wᵢᵢᵢᵢᵢ vary between −1,000 and 1,000.

Aspect 9. The process of Aspect 8, wherein a timespan within which a certain set of equations and related coefficients are used to predict unit performance, N, is calculated using Equation (4):

$$N = ( {\sum }_{0}^{t} {M}_{[ Specie A ]} ) \cdot {W}_{i} + ( {\sum }_{0}^{t} \frac{{M}_{[ Specie A ]}}{{M}_{[ Feed ]}} ) \cdot {W}_{ii} + ( {\sum }_{0}^{t} \frac{{M}_{[ Specie A ]}}{{M}_{[ Specie B ]}} ) \cdot {W}_{iii} + ( {\sum }_{0}^{t} \frac{{M}_{[ Specie A ]} \cdot {M}_{[ Specie C ]}}{{M}_{[ Feed ]}} ) \cdot {W}_{iiii} + ( {\sum }_{0}^{t} \frac{{M}_{[ Specie A ]} \cdot {M}_{[ Specie C ]}}{{M}_{[ Specie B ]}} ) \cdot {W}_{iiiii} \qquad ( 4 )$$

Aspect 10. The process of Aspect 9, wherein an updated timespan within which a certain set of equations and related coefficients are used to predict unit performance, N′, is calculated using Equation (5):

$${N}^{′} = \frac{N}{{M}_{Active Material}} \cdot X + \frac{N}{{M}_{Inert}} \cdot Y + \frac{N}{( {M}_{Active material} + {M}_{Inert} )} \cdot Z \qquad ( 5 )$$

wherein
M_{Active Material} is a mass of material playing an active role in either transforming alkane molecules to olefins, playing an active role in the heat balance of dehydrogenation reactors, or a combination thereof,
M_{Inert} is a mass of material not playing an active role in chemical transformation or heat balance of dehydrogenation reactors,
N is the timespan within which the certain set of equations and related coefficients are used to predict unit performance calculated using Equation (4), and
X, Y and Z are coefficients varying between 0 and 1.

Aspect 11. The process of any of the preceding aspects, wherein determining a predicted change in production of the product of the chemical process using the process performance model comprises determining more than one predicted change in production of the product of the chemical process using the process performance model.

As will be appreciated by one skilled in the art, aspects may be embodied as a system, method or computer program product. Accordingly, aspects may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wire line, optical fiber cable, RF, etc., or any suitable combination of the foregoing.

Computer program code for carrying out operations for aspects may be written in any combination of one or more programming languages, including an object oriented programming language such as Java, Smalltalk, C++ or the like and conventional procedural programming languages, such as the “C” programming language or similar programming languages. The program code may execute entirely on the user' s computer, partly on the user' s computer, as a stand-alone software package, partly on the user' s computer and partly on a remote computer or entirely on the remote computer or server. In the latter scenario, the remote computer may be connected to the user' s computer through any type of network, including a local area network (LAN) or a wide area network (WAN), or the connection may be made to an external computer (for example, through the Internet using an Internet Service Provider).

Aspects are described above with reference to flowchart illustrations and/or block diagrams of methods, apparatus (systems) and computer program products according to embodiments. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods and computer program products according to various embodiments. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The processes and methods can alternatively comprise, consist of, or consist essentially of, any appropriate materials, steps, or components herein disclosed. The processes and methods can additionally, or alternatively, be formulated so as to be devoid, or substantially free, of any materials (or species), steps, or components, that are otherwise not necessary to the achievement of the function or objectives of the processes and methods.

The terms “a” and “an” and “the” do not denote a limitation of quantity and are to be construed to cover both the singular and the plural, unless otherwise indicated herein or clearly contradicted by context. “Or” means “and/or” unless clearly stated otherwise. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments. A “combination thereof” is open and includes any combination comprising at least one of the listed components or properties optionally together with a like or equivalent component or property not listed.

Unless specified to the contrary herein, all test standards are the most recent standards in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

Although the processes and methods of the present disclosure have been described with reference to exemplary embodiments thereof, the present disclosure is not limited to such exemplary embodiments and/or implementations. Rather, the processes and methods of the present disclosure are susceptible to many implementations and applications, as will be readily apparent to persons skilled in the art from the disclosure hereof. The present disclosure expressly encompasses such modifications, enhancements and/or variations of the disclosed embodiments. Since many changes could be made in the above construction and many widely different embodiments of this disclosure could be made without departing from the scope thereof, it is intended that all matter contained in the drawings and specification shall be interpreted as illustrative and not in a limiting sense. Additional modifications, changes, and substitutions are intended in the foregoing disclosure. Accordingly, it is appropriate that the appended claims be construed broadly and in a manner consistent with the scope of the disclosure.

1. A process for operating a chemical process comprising:
deriving coefficients for a process performance model from historical feed data and historical production data;
formulating the process performance model using the coefficients;
determining a predicted change in production of a product of the chemical process using the process performance model; and
changing a processing parameter of the chemical process based on economic data and the predicted change in production of the product of the chemical process.

2. The process of claim 1, wherein the economic data comprises contribution margin from sales of the product of the chemical process; earnings before interest, taxes, depreciation, and amortization; maintenance cost; catalyst cost; or a combination thereof.

3. The process of claim 1, wherein changing the processing parameter comprises changing the catalyst and catalyst bed configuration.

4. The process of claim 1, further comprising updating the process performance model.

5. The process of claim 4, wherein updating the process performance model comprises using additional feed data and additional production data.

6. The process of claim 4, wherein updating the process performance model comprises using updated economic data, additional economic data, or a combination thereof.

7. The process of claim 1, wherein the chemical process comprises alkane dehydrogenation.

8. The process of claim 7, wherein the coefficients comprise m, m₁, m₂, Mᵢ, Wᵢ, Wᵢᵢ, Wᵢᵢ, Wᵢᵢᵢᵢ, and Wᵢᵢᵢᵢᵢ in Equations (1), (3), and (6):
$$\frac{daily i = {C}_{4 PROD}}{daily i - {C}_{4 FED}} ( t ) = \frac{daily i = {C}_{4 PROD}}{daily i - {C}_{4 FED}} ( {t}_{0} ) - {m}_{1} \cdot {t}^{2} ( DOS ) + {m}_{2} \cdot t ( DOS ) \qquad ( 1 )$$
$$\frac{daily i = {C}_{4 PROD}}{daily i - {C}_{4 FED}} ( t ) = \frac{daily i = {C}_{4 PROD}}{daily i - {C}_{4 FED}} ( {t}_{0} ) - m \cdot t ( DOS ) . \qquad ( 3 )$$
$${M}_{i} = {W}_{i} \cdot \frac{d}{dt} {M}_{[ Specie A ]} + {W}_{ii} \cdot \frac{d}{dt} ( \frac{{M}_{[ Specie A ]}}{{M}_{[ Feed ]}} ) + {W}_{iii} \cdot \frac{d}{dt} ( \frac{{M}_{[ Specie A ]}}{{M}_{[ Specie B ]}} ) + {W}_{iiii} \cdot \frac{d}{dt} ( \frac{{M}_{[ Specie A ]} \cdot {M}_{[ Specie C ]}}{{M}_{[ Feed ]}} ) + {W}_{iiiii} \cdot \frac{d}{dt} ( \frac{{M}_{[ Specie A ]} \cdot {M}_{[ Specie C ]}}{{M}_{[ Specie B ]}} ) \qquad ( 6 )$$
wherein
i=C_{4PROD} is amount of isobutene produced,
t is a certain time,
i-C_{4FED} is amount of isobutane fed to a dehydrogenation unit,
F is averaged daily flow rate to be multiplied by i=C_{4PROD}/i-C_{4FED} at t,
DOS is Days On Stream,
M is daily mass, and
Wᵢ, Wᵢᵢ, Wᵢᵢᵢ, Wᵢᵢᵢᵢ, and Wᵢᵢᵢᵢᵢ vary between −1,000 and 1,000.

9. The process of claim 8, wherein a timespan within which a certain set of equations and related coefficients are used to predict unit performance, N, is calculated using Equation (4):
$$N = ( {\sum }_{0}^{t} {M}_{[ Specie A ]} ) \cdot {W}_{i} + ( {\sum }_{0}^{t} \frac{{M}_{[ Specie A ]}}{{M}_{[ Feed ]}} ) \cdot {W}_{ii} + ( {\sum }_{0}^{t} \frac{{N}_{[ Specie A ]}}{{N}_{[ Specie B ]}} ) \cdot {W}_{iii} + ( {\sum }_{0}^{t} \frac{{N}_{[ Specie A ]} \cdot {M}_{[ Specie C ]}}{{M}_{[ Feed ]}} ) \cdot {W}_{iiii} + ( {\sum }_{0}^{t} \frac{{M}_{[ Specie A ]} \cdot {M}_{[ Specie C ]}}{{M}_{[ Specie B ]}} ) \cdot {W}_{iiiii} . \qquad ( 4 )$$

10. The process of claim 9, wherein an updated timespan within which a certain set of equations and related coefficients are used to predict unit performance, N′, is calculated using Equation (5):
$${N}^{′} = \frac{N}{{M}_{Active Material}} \cdot X + \frac{N}{{M}_{Inert}} \cdot Y + \frac{N}{( {M}_{Active material} + {M}_{Inert} )} \cdot Z \qquad ( 5 )$$
wherein
M_{Active Material} is a mass of material playing an active role in either transforming alkane molecules to olefins, playing an active role in the heat balance of dehydrogenation reactors, or a combination thereof,
M_{Inert} is a mass of material not playing an active role in chemical transformation or heat balance of dehydrogenation reactors,
N is the timespan within which the certain set of equations and related coefficients are used to predict unit performance calculated using Equation (4), and
X, Y and Z are coefficients varying between 0 and 1.

11. The process of claim 1, wherein determining a predicted change in production of the product of the chemical process using the process performance model comprises determining more than one predicted change in production of the product of the chemical process using the process performance model.
